(19) United States
(12) Patent Application Publication
(10) Pub. No.: US 2019/0108902 A1
(43) Pub. Date: Apr. 11, 2019

(54) A REMOTE MEDICAL IMAGING SYSTEM MANAGEMENT ARRANGEMENT

(71) Applicant: KONINKLIJKE PHILIPS N.V., EINDHOVEN (NL)

(72) Inventors: VINAY PARTHAN, BEST (NL); SATHISH KUMAR BALAKRISHNAN, BANGALORE (IN)

(21) Appl. No.: 16/089,070

(22) PCT Filed: Mar. 27, 2017
(86) PCT No.: PCT/EP2017/057141

§ 371 Date: Sep. 27, 2018

(30) Foreign Application Priority Data
Apr. 4, 2016 (IN) 201641011782
May 17, 2016 (EP) 16169848.5

(51) Int. Cl. G16H 30/20; G16H 10/60; G06F 16/51

(52) U.S. Cl. CPC G16H 30/20; G06F 16/51; G16H 10/60

(57) ABSTRACT

It is an object of the invention to provide for a system that can help in using medical imaging systems in an improved way by reducing operating costs of the medical imaging systems or by improving image quality. This object is achieved by a medical imaging system management arrangement comprising multiple medical imaging systems configured to acquire medical imaging data according to an imaging protocol. The medical imaging system management arrangement also comprises a central unit connected to the multiple medical imaging systems via a data link. The central unit comprises a database comprising imaging protocols, wherein the central unit is configured to distribute imaging protocols to the multiple medical imaging systems and wherein the remote medical imaging system management arrangement is configured such that adaptation of the imaging protocols is restricted or impossible at the locations of the multiple medical imaging systems.

### FIELD OF THE INVENTION

The invention relates to the field of diagnostic imaging and more specifically to the field of magnetic resonance imaging.

### BACKGROUND OF THE INVENTION

US2003/0181804 describes a plurality of diagnostic scanners that share access to a remote communual processing center that performs reconstruction and post reconstruction processing for various modalities. Each of the diagnostic scanners submits a data set to the remote center electronically over the lines. The reconstructed image representations are sent electronically to the address that sent them.

### SUMMARY OF THE INVENTION

It is an object of the invention to provide for a system that can help in using medical imaging systems in an improved way by reducing operating costs of the medical imaging systems or by improving image quality. This object is achieved by a medical imaging system management arrangement according to claim 1. At present many radiology departments use their own version of an MRI imaging protocol. This may negatively affect image quality and efficiency. By restricting adaptation of the imaging protocols image quality and/or efficiency may be improved. Also a level of standardization may be increased.

In one aspect, the invention relates to a remote medical imaging system management arrangement comprising:
multiple medical imaging systems configured to acquire medical imaging data according to an imaging protocol;
a central unit connected to the multiple medical imaging systems via a data link, wherein the central unit comprises a database comprising imaging protocols, wherein the central unit is configured to distribute imaging protocols to the multiple medical imaging systems and wherein the remote medical imaging system management arrangement is configured such that adaptation of the imaging protocols is restricted or impossible at the locations of the multiple medical imaging systems.

According to embodiments of the invention the distributed imaging protocols are standardized and are similar for the multiple medical imaging systems. This is advantageous especially when access to well-trained personel is restricted. By the use of standardized imaging protocols overall image quality may be improved. Further, this embodiment is espcially advantageous if the central unit is configured to at least partly control one or more of the medical imaging systems. Standardization of the imaging protocols will make it easier for a lead technician at the location of the central unit to be involved in the operation of multiple medical imaging systems at the same time.

According to further embodiments of the invention the remote medical imaging system management arrangement is configured to allow adaptations of an imaging protocol for a current patient at one or more of the locations of the respective multiple medical imaging systems, but the remote medical imaging system management arrangement is also configured to prohibit to save the adaptations for use in a future patient. This embodiment is advantageous, because it allows more flexibility at the sites of the medical imaging systems. This could be beneficial in situations wherein a fully standardized protocol is not suitable for a certain patient. Reasons for this could for example be the size of the patient, certain medical conditions, medical artifacts and so on. By prohibiting that the adaptation can be saved the standardization can be maintained, which may result in overall improved image quality.

According to further embodiments the remote medical imaging system management arrangement is configured to allow adaptations of an imaging protocol at the location of one or more of the medical imaging systems and to save the adapted imaging protocol to the database as a new version of the imaging protocol. This embodiment is advantageous, because at certain locations, the personnel may have better insights on how a protocol can be improved in general or for a specific patient group. By allowing to save adaptations as a new version into the database, other locations may benefit from these adapted and potentially improved protocols. There are several ways on how one could deal with those new versions in the database. A few examples will be given here. First of all, it could be decided that the adaptations are not an improvement and the version could be deleted or ignored. If the new version is considered an improvement, the new version could replace the previous version for all locations. Also the new version could be an improvement for specific patient groups and/or disease, e.g. patients of a certain size or patients having a certain medical condition. In these cases the new version could be used for the specific patient groups, whereas the previous version could still be used for other patient groups. This embodiment is advantageous, because it allows improvements but at the same time it may help to guarantee an certain overall image quality.

According to further embodiments of the invention, the database comprises a selection of imaging protocols for which use a license is required and wherein the remote medical imaging system management arrangement is configured to only distribute the selection of imaging protocols to the medical imaging systems for which the license is available. This embodiment is advantageous, because it may be an easy way to perform license management. Also this embodiment could be useful where a customer wants to do a “Try & Buy”. The customer could download a temporary license to Try an application for some period of time and then decide to Buy.

According to further embodiments of the invention, the license is a floating license and the remote medical imaging system management arrangement is configured to allow a first medical imaging system to use a license if the license is not in use by a second medical imaging system. This embodiment is advantageous, because it may provide more flexibility to the users of the medical imaging systems on the use of the license.

According to further embodiments of the invention, the database comprises information on patient conditions and the remote medical imaging system management arrangement is further configured to automatically select an imaging protocol for an individual patient based on the individual patient's conditions. The database may be a realtime information sniffed through a HL7/DICOM network whenever the patient gets registered. This embodiments could have one of the following advantages. First of all, it may improve patient safety, especially if the remote medical imaging system management arrangement prohibits the use of certain imaging protocols for patients suffering from certain conditions. Such conditions could for example be a presence of a metal implant, use of certain medication or allergies. Secondly, specific imaging protocols for specific patients may improve image quality for these patients.

These and other aspects of the invention will be apparent from and elucidated with reference to the embodiments described hereinafter.

### BRIEF DESCRIPTION OF THE DRAWINGS

FIG. 1.1 diagrammatically shows a remote medical imaging system management arrangement according to embodiments of the invention.

### DETAILED DESCRIPTION OF EMBODIMENTS

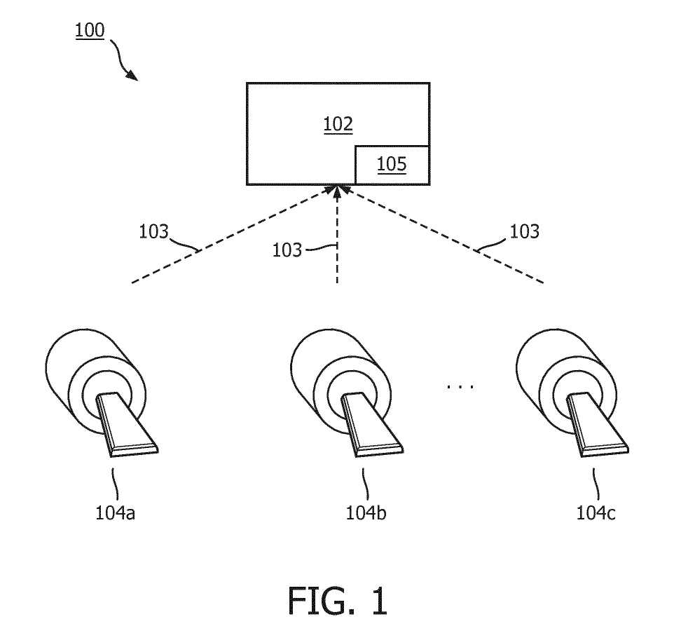

FIG. 1 diagrammatically shows a remote medical imaging system management arrangement 100 according to embodiments of the invention. The remote medical imaging system management arrangement comprises multiple medical imaging systems 104a, 104b, 104c. In FIG. 1 the multiple medical imaging systems are MRI systems. The multiple medical imaging systems are configured to acquire medical imaging data according to an imaging protocol. The remote medical imaging system management arrangement comprises a central unit 102 connected to the multiple medical imaging systems via a data link 103. The central unit 102 comprises a database 105 comprising imaging protocols. The central unit is configured to distribute imaging protocols to the multiple medical imaging systems via the datalink 103. The remote medical imaging system management arrangement is configured such that adaptation of the imaging protocols is restricted or impossible at the locations of the multiple medical imaging systems.

The restriction in adaptation could be implemented in several ways. It could imply that adaptation of protocols is completely prohibited at one or more of the locations of the medical imaging systems. Also it could be allowable to adapt one or more of the protocols at one or more of the locations of the medical imaging systems for a current patient, whereby saving the adaptations for use in a future patient is prohibited. Also it could be allowable to adapt one or more of the protocols at one or more of the locations of the medical imaging systems and to save those the adapted protocol as a new version of the protocol. Such new version can be saved in database 105. An operator at the site of the central unit may have the permission to decide what will happen with the new version of the protocol (e.g. whether it will replace a current version, whether it will be deleted, whether it will be a new and dedicated protocol for a specific patient group and/or disease.)

The database 105 could comprise a selection of imaging protocols for which use a license is required. This license could be a floating license. The remote medical imaging system management arrangement could be configured to only distribute the selection of imaging protocols to the medical imaging systems for which the license is available.

The remote medical imaging system management arrangement could also comprise a database with patient information. This database could comprise information on patient conditions. The remote medical imaging system management arrangement could be configured to automatically select an imaging protocol for an individual patient based on the individual patient's conditions.

Whilst the invention has been illustrated and described in detail in the drawings and foregoing description, such illustrations and description are to be considered illustrative or exemplary and not restrictive; the invention is not limited to the disclosed embodiments.

1. A remote medical imaging system management arrangement comprising:
multiple medical imaging systems configured to acquire medical imaging data according to an imaging protocol;
a central unit connected to the multiple medical imaging systems via a data link, wherein the central unit comprises a database comprising imaging protocols, wherein the central unit is configured to distribute imaging protocols to the multiple medical imaging systems and wherein the remote medical imaging system management arrangement is configured such that adaptation of the imaging protocols is restricted at the locations of the multiple medical imaging systems.

2. The remote medical imaging system management arrangement of claim 1, wherein the distributed imaging protocols are standardized using standardized imaging protocols and are similar for the multiple medical imaging systems.

3. The remote medical imaging system management arrangement of claim 1, configured to allow adaptations of an imaging protocol for a current patient at one or more of the locations of the respective multiple medical imaging systems, but wherein the remote medical imaging system management arrangement is also configured to prohibit to save the adaptations for use in a future patient

4. The remote medical imaging system management arrangement of claim 1, configured to allow adaptations of an imaging protocol at the location of one or more of the medical imaging systems and to save the adapted imaging protocol to the database as a new version of the imaging protocol.

5. The remote medical imaging system management arrangement of claim 1, wherein the database comprises a selection of imaging protocols for which use a license is required and wherein the remote medical imaging system management arrangement is configured to only distribute the selection of imaging protocols to the medical imaging systems for which the license is available.

6. The remote medical imaging system management arrangement of claim 5, wherein the remote medical imaging system management arrangement is configured to allow a first medical imaging system to use a license if the license is not in use by a second medical imaging system.

7. The remote medical imaging system management arrangement of claim 5 further comprising a database comprising information on patient conditions, wherein the remote medical imaging system management arrangement is further configured to automatically select an imaging protocol for an individual patient based on the individual patient's conditions.
